(19) United States
(12) Patent Application Publication
(10) Pub. No.: US 2006/0035279 A1
(43) Pub. Date: Feb. 16, 2006

(54) Anti-GPE antibodies, their uses and assays for weakly immunogenic molecules

(75) Inventors: David Charles Batchelor, Auckland (NZ); Gregory Brian Thomas, Perth (AU); Bernhard Hermann Heinrich Breier, Auckland (NZ)

Correspondence Address: FLIESLER MEYER, LLP, FOUR EMBARCADERO CENTER, SUITE 400, SAN FRANCISCO, CA, 94111, US

(73) Assignee: Neuren Pharmaceuticals Ltd., Auckland (NZ)

(21) Appl. No.: 10/971,359

(22) Filed: Oct. 22, 2004

Related U.S. Application Data
(63) Continuation-in-part of application No. 10/100,515, filed on Mar. 14, 2002.
(60) Provisional application No. 60/276,796, filed on Mar. 16, 2001.

(51) Int. Cl. G01N 33/53; C07K 16/18

(52) U.S. Cl. 435/7.1; 530/388.1

(57) ABSTRACT

Anti-GPE antibodies are provided that can be used to detect the presence of GPE in a sample. GPE antibodies can be raised against conjugated GPE to produce antibodies that are specific for a GPE that has been derivatized using a similar coupling chemistry as was used for making the GPE conjugate. To detect GPE in a sample, the sample is exposed to a derivatizing agent to produce a derivatized GPE that is recognized by the anti-GPE antibody. Using similar strategies, antibodies can be raised against other weakly immunogenic molecules (WIMs) that can be the basis for assays and kits for assaying for WIMs.

### PRIORITY CLAIM

This application is a Continuation-In-Part of U.S. Ser. No. 10/100,515, titled “Anti-GPE Antibodies, Their Uses, and Analytical Methods for GPE,” Gregory Brian Thomas, Bernhard Hermann Heinrich Breier, and David Charles Batchelor, inventors, filing date Mar. 24, 2002 (Attorney Docket No: NRNZ 1016 US1 DBB), which claimed priority to U.S. Provisional Patent Application Ser. No. 60/276,796, filed Mar. 16, 2001, titled “Analytical Methods for the Detection of GPE,” Gregory Brian Thomas, Bernhard Hermann Heinrich Breier and David Charles Batchelor, inventors, (now abandoned; Attorney Docket No: NRNZ 1016 US0 DBB). Both of the above applications are incorporated herein fully by reference.

### FIELD OF THE INVENTION

This invention relates to anti-GPE antibodies and their uses, to analytical methods for GPE and methods for sustained administering of GPE to animals for treatment of neurodegenerative disorders. This invention also relates to methods for assaying for weakly immunogenic molecules in samples.

### BACKGROUND

Neurodegenerative conditions are sources of continued morbidity and mortality in humans afflicted with one or of a variety of acute and/or chronic conditions. Ischemia, hypoxia, stroke, Alzheimer's disease, Parkinsons' disease, and other conditions can lead to progressive loss of neural function. Although some information about the effects of such conditions is known, there has been little improvement in the ways in which neural cell and/or glial cells die or degenerate in these conditions. Further, there have been few advances in the treatment of neurodegenerative conditions.

EP 0 366 638 discloses GPE (a tri-peptide consisting of the amino acids Gly-Pro-Glu) and its di-peptide derivatives Gly-Pro and Pro-Glu. EP 0 366 638 discloses that GPE is effective as a neuromodulator and is able to alter the release of neurotransmitter from neurons exposed to potassium. EP 0 366 638 also discloses reflex-potentiating effects of GPE in animals from which the brains had been removed.

WO95/172904 discloses that GPE has neuroprotective properties and that administration of GPE can reduce damage to the central nervous system (CNS) by the prevention or inhibition of neuronal and glial cell death.

WO 98/14202 discloses that administration of GPE can increase the effective amount of choline acetyltransferase (ChAT), glutamic acid decarboxylase (GAD), and nitric oxide synthase (NOS) in the central nervous system (CNS).

WO99/65509 and U.S. application Ser. No. 09/719,459 disclose that increasing the effective amount of GPE in the CNS, such as by administration of GPE, GP or PE, can increase the effective amount of tyrosine hydroxylase (TH) in the CNS for increasing TH-mediated dopamine production in the treatment of diseases such as Parkinson's disease.

WO02/16408 discloses GPE analogs capable of inducing a physiological effect equivalent to GPE within a patient. The applications of the GPE analogs include the treatment of acute brain injury and neurodegenerative diseases, including but not limited to, injury or disease in the CNS.

Although GPE has been shown to be neuroprotective, the pharmacokinetics of GPE has been poorly understood, and therefore, suitable therapeutic methods of administering GPE are poorly known. Although statistically significant neuroprotective effects of GPE have been observed, the variability of responses to GPE indicates that substantial, unknown factor(s) affect neuroprotective responses to GPE. In particular, identification of these factor(s) has led to new and unexpected improvements in the therapeutic administration of GPE.

The disclosures of these and other documents referred to in this application (including in the Figures) are incorporated herein by reference.

### SUMMARY OF INVENTION

One embodiment of this invention includes antibodies against GPE (“anti-GPE antibodies”), including antibodies to derivatized GPE, which recognizes a derivatized GPE.

Another embodiment of the invention includes a radioimmunoassay methods for the measurement of GPE using the anti-GPE antibodies of the first embodiment and kits for the same.

Other embodiments include radioimmunoassay methods for detecting the presence of weakly immunogenic molecules comprising making an antibody to a conjugated molecule, derivatizing the molecule present in a sample, and then exposing the derivatized molecule to the antibody made against the conjugated molecule.

Further embodiments include kits for radioimmunoassay for GPE and weakly immunogenic molecules.

### BRIEF DESCRIPTION OF THE FIGURES

FIG. 1 is a graph showing a radioimmunoassay displacement curve showing competitive displacement by unlabeled GPE or ¹²⁵I-labeled YGP binding to the CK5 antibody.

FIG. 2 is a graph is a radioimmunoassay displacement curve showing competitive displacement by unlabeled Bolton Hunter derivatized GPE of ¹²⁵I-labeled Bolton and Hunter derivatized YGPE binding to the CK5 antibody and lack of cross reactivity with the Bolton and Hunter derivatized forms of glycine, proline, glutamate, insulin-like growth-factor 1 (IGF-1), gly-pro and urea.

FIG. 3 is a graph depicting comparison of the displacement of radio-labeled ¹²⁵I-YGPE by GPE with the displacement of ¹²⁵I-Bolton and Hunter derivatized GPE by Bolton and Hunter derivatized GPE.

FIG. 4 is a graph depicting standard curves of □, GPE (665.7 ng/ml); ⋄, KYFGGPE (2.153 ng/ml) and ◯, YGPE (3.922 ng/ml) against ¹²⁵I-YGPE and ●, GPE (408 ng/ml) and ▪, BH-GPE (0.175 ng/ml) against ¹²⁵I-BH-GPE using CK-5 antisera at 1/18000. IC₅₀ values in parenthesis.

FIG. 5 is a graph showing assay specificity of ¹²⁵I-BH-GPE for Bolton and Hunter derivatized forms of GPE metabolites in comparison to BH-GPE(▪). Gly (▴), Pro (⋄), Glu (♦) and Gly-Pro (●) did not cross react with a 1/18000 dilution of CK-5 antibody when using 125I-BH-GPE as tracer. Pro-Glu (□) had a cross-reactivity of 0.17%.

FIG. 6 is a graph showing detection of GPE in plasma. Extracted plasma (●) or buffer(▪) samples were spiked with GPE. Samples were then derivatized with Bolton and Hunter reagent and then measured by radioimmunoassay. GPE in plasma had a small change of IC₅₀ from 0.175 to 0.652 ng/ml compared to buffer, but was otherwise parallel indicating there were no cross-reactive compounds in plasma.

FIG. 7 depicts a graph showing an HPLC elution pattern of GPE standard (−) and GPE immuno-reactivity from an extract of plasma spiked with GPE and resolved by HPLC (▪). Samples (100 μl) were injected onto a C₁₈ Aquapore column as described in the methods and the fractions radio-immunoassayed. RIA analysis of the fractions identified fraction 12 and 13 as having the highest immuno-reactivity which matched the elution time for the GPE standard of 12 min as measured by UV absorbance at 200 nm

FIGS. 8A, 8B and 8C depict graphs showing the measurement of GPE in plasma using the CK5 antibody following intravenous administration of GPE at 3 mg/kg (FIG. 8A), 30 mg/kg (FIG. 8B) and 100 mg/kg (FIG. 8C).

FIG. 9 is a graph showing an rpHPLC chromatogram showing the resolution of GPE in plasma following derivatization by AccQTag® reagent.

FIGS. 10A, 10B, 10C and 10D are graphs depicting rpHPLC chromatograms showing detection of GPE in blood. A baseline measurement is shown in FIG. 10A. FIGS. 10B-10D depict GPE and degradation products in plasma following 1 min (FIG. 10B), 2 min (FIG. 10C), and 8 min (FIG. 10D) following intravenous administration of 30 mg/kg GPE.

FIG. 11 is a graph showing an rpHPLC chromatogram showing the resolution of tritiated GPE in plasma.

### DETAILED DESCRIPTION OF INVENTION

In a first aspect, this invention is antibodies against GPE (“anti-GPE antibodies”). These anti-GPE antibodies may be prepared by immunization of animals (e.g., rabbits) with immunogens containing GPE conjugated to an antigen such as keyhole limpet hemocyanin, as described below to produce a GPE conjugate. Such conjugates can desirably have molecular weights of greater than 200 Daltons. A polyclonal anti-GPE antiserum, which we refer to as CK5 antibody, specifically recognises GPE and binds GPE with a high titer. In preferred embodiments of the invention, the antibodies to GPE are chracterized by the ability to specifically bind to GPE using ¹²⁵I-YGPE tracer with a final titer of at least about 1:600. In embodiments of the invention, the anti-GPE antibodies have the ability to bind specifically to GPE in normal tissues; or have the ability to bind specifically to GPE in diseased or injured tissue. In a most preferred embodiment of the invention, the anti-GPE antibodies have the ability to bind GPE in diseased or injured tissue of the central or peripheral nervous system. In another embodiment of the invention, the anti-GPE antibodies have the ability to specifically bind to Bolton and Hunter derivatized GPE using ¹²⁵I-Bolton and Hunter derivatized GPE tracer with a final titer of at least about 1:18,000.

Anti-GPE antibodies may be monoclonal or polyclonal. Methods for making antibodies are generally well known in the art and need not be described in detail here. However, to produce antibodies against weakly immunogenic molecules (WIMs), new methods are used as described further herein.

Anti-GPE antibodies find use in determining the pharmacokinetics and pharmacodynamics of GPE and GPE-related compounds (GPE analogs); an in assays to determine the neuroprotective concentration of GPE in blood and CSF required in the treatment of a disease or in the treatment of injury. Examples of such uses are found in U.S. Provisional Patent Application Ser. No. 60/513,851, filed Oct. 23, 2003, U.S. Provisional Patent Application Ser. No. 60/515,397, filed Oct. 28, 2003, U.S. Provisional Patent Application Ser. No. 60/553,688 filed Mar. 16, 2003 and PCT International Patent Application titled “Neuroprotective Effects of Gly-Pro-Glu Following Intravenous Injection, Jian Guan, Gregory Brian Thomas, David Charles Batchelor and Peter D. Gluckman, inventors, filed concurrently (Attorney Docket No: NRNZ 1052 WO0). Each of the afore mentioned patent applications is incorporated herein fully by reference. In preferred embodiments of the invention, anti-GPE antibodies find use in assays to determine the neuroprotective concentration of GPE in blood and CSF required in the treatment of Parkinson's disease, multiple sclerosis, Alzheimer's disease, Huntington's disease, peripheral neuropathy, stroke, cardiac artery bypass graft surgery, ischemic brain injury, hypoxic brain injury, traumatic brain injury, and in the treatment of pancreatic disease including type 1 and type 2 diabetes. Further embodiments of the invention provide methods for the use of anti-GPE antibodies in the in vitro evaluation of GPE function. Such methods include evaluation of the effects of in vitro administration of GPE in the presence and in the absence of anti-GPE antibodies.

In a second aspect, this invention is a radioimmunoassay method for the measurement of GPE using the anti-GPE antibodies of the first aspect of this invention, as described herein. The radioimmunoassay method allows for the selective quantitation of GPE in body fluids, (e.g., blood, serum, cerebrospinal fluid, and urine) and in body tissues. The level of GPE may be a suitable marker of drug efficacy and/or effective dosing. In one embodiment of the invention, a radioimmunoassay kit comprises an anti-GPE antibody, a GPE standard, as assay buffer, a GPE compound (e.g., YGPE) for iodination, and a second antibody or a precipitated antibody, for example an antibody precipitated with polyethylene glycol (PEG). In another embodiment the kit comprises an anti-GPE antibody, a GPE standard, an assay buffer, tritiated GPE, and a second antibody or a precipitated antibody. In a further embodiment, a radioimmunoassay kit comprises an anti-GPE antibody, a GPE standard, Bolton and Hunter reagent (N-succinimidyl-3-[4-hydroxyphenyl] propionate), a derivatizing buffer, an assay buffer, Bolton and Hunter derivatized GPE (BH-GPE) for iodination, and a second or precipitating antibody, for example an antibody precipitated with polyethylene glycol.

In a third aspect, this invention is methods of reverse phase high-performance liquid chromatography (“rpHPLC”) that accurately resolves and quantitates GPE and related compounds. Two methods are described herein, the first using derivatization of the amino groups with AccQTag® reagent, 6-aminoquinolyl-N-hydroxysuccinimidyl carbamate in acetonitrile and borate buffer, and the second, for the measurement of radioactive (e.g., tritiated) GPE, using a Hypercarb® column with no derivatization, and detection of the radioactivity in the eluate. The level of GPE may be a suitable marker of drug efficiency and/or effective dosing. In one embodiment, an rpHPLC assay kit comprises a GPE standard, 6-aminoquinolyl-N-hydroxysuccinimidyl carbamate and derivatizing buffer, a column, a column running buffer. In another embodiment, an rpHPLC assay kit comprises a radioactive GPE standard, a column, and column running buffer.

In fourth aspect this invention, therapeutic methods using the anti-GPE antibodies are described. This invention is methods for the extension of the half-life of GPE in vitro and in vivo comprising co-administration of GPE with anti-GE antibodies effective that a significant fraction of the GPE is bound to the anti-GPE antibody a significant fraction of the time, thereby protecting the GPE from degradation, non-specific binding and metabolic modification and clearance. In further embodiments of the invention, antibodies to GPE may be used as non-blocking antibodies to modulate concentrations of GPE. In particular, antibodies to GPE may be used as non-blocking antibodies to modulate the free concentrations of GPE in vivo and in vitro.

In a fifth aspect, this invention is method for the purification of the GPE receptor, comprising the use of the anti-GPE antibodies of the first aspect of this invention, as described herein. In these methods, tissues, suspensions and solutions comprising GPE receptors contact surfaces, substrates, and solutions comprising GPE, effective to bind the GPE to the GPE receptors; and such surfaces, substrates, and solutions are subsequently contacted with anti-GPE antibodies so that the anti-GPE antibody binding to the GPE that is bound to GPE receptors is effective to aid in the purification of the GPE receptors.

In a sixth aspect, this invention relates to use of an anti-GPE antibody for the manufacture of a medicament for extending the half-life of GPE or modulating the free concentration of GPE. The anti-GPE antibody of the invention may be co-administered with GPE.

In a seventh aspect, the invention related to a composition for extending the half-life of GPE or modulating the free concentration of GPE, which composition comprises an anti-GPE antibody of the invention and at least one pharmaceutically acceptable excipient administered in vivo. The composition may further comprise GPE. The composition may be administered in vivo.

In general, the anti-GPE antibody may be administered by one of the following routes: directly to the central nervous system, oral, topical, systemic (e.g., transdermal, intranasal, or by suppository), parenteral (e.g., intramuscular, subcutaneous, or intravenous injection), by implantation and by infusion through such devices as osmotic pumps, transdermal patches and the like. Compositions may take the form of tablets, pills, capsules, semisolids, powders, sustained release formulation, solutions, suspensions, elixirs, aerosols or any other appropriate compositions; and comprise at least anti-GPE antibody in combination with at least one pharmaceutically acceptable excipient. Suitable excipients are well known to persons of ordinary skill in the art, and they, and the methods of formulating the compositions, may be found in such standard references as Gennaro, ed. (2000), “Remington: The Science and Practice of Pharmacy”, 20^{th} ed., Lippincott, Williams & Wilkins, Philadelphia Pa. Suitable liquid carriers, especially for injectable solutions, include water, aqueous saline solution, aqueous dextrose solution, and the like, with isotonic solutions being preferred for intravenous administration.

The anti-GPE antibody in combination with GPE can be administered directly to the central nervous system. This route of administration can involve, for example, lateral cerebroventricular injection, focal injection, or a surgically inserted shunt into the lateral cereberal ventricle.

In an additional aspect, this invention includes methods for producing antibodies against WIMs and kits suitable for use in assaying for WIMs in samples, including biological samples, such as plasma, cerebrospinal fluid (CSF) and other bodily fluids. Additionally, such anti-WIM antibodies can be suitable for detecting WIMs in tissues or in pharmaceutical preparations to quantify the amount of a WIM used for pharmaceutical purposes.

The following non-limiting Examples illustrate this invention. All animal experimental protocols were conducted in accordance with guidelines approved by the University of Auckland Ethics Committee.

When administered by intracerebroventricular (i.c.v.), intraperitoneal (i.p.) and intravenous (i.v.) injections following acute ischemic brain injury, GPE has been shown to be neuroprotective in vivo reducing both cortical damage and neuronal loss in the CA1-2 subregions of the hippocampus (2, 3, 5). Furthermore, GPE had a stimulatory effect on the potassium induced release of acetylcholine from rat cortical slices (4).

Although statistically significant neuroprotective effects of GPE have been observed, we previously found an unexplained variability in responses to GPE among different areas of the brain, and among different animals. Herein, we have identified a source of the variability in response, and by taking that source into effect, we have produced a therapeutic regimen that increases the magnitude of the neuroprotective effect, decreases the variability between different areas of the brain, and decreases the overall variability between animals. Therefore, certain embodiments of this invention include new radioimmunoassay procedures for quantifying weakly immunogenic materials, such as GPE. Using such methods, we measured the metabolism of GPE in plasma and cerebrospinal fluid (CSF), and surprisingly found that GPE is rapidly metabolised in plasma. The measured half-life (t½) is on the order of 2-5 minutes.

As a result of this new finding, we developed in vivo protocols for administration of GPE and tested the new protocols in animals subjected to neurodegenerating conditions (hypoxia/ischemia). We unexpectedly found that with sustained infusion, the magnitude of the neuroprotective effect of GPE was substantially increased, and that the variability in responses was markedly reduced.

Methods for Quantifying GPE in Biological Samples

Knowing pharmacokinetic properties of GPE can permit the rational design of dosing regimens to more effectively treat conditions for which GPE is useful. To carry out pharmacokinetic studies of GPE, we first needed a reliable, sensitive assay for GPE in plasma and other body fluids. However, many small molecules by themselves are only weak immunogens, so we developed a strategy for increasing the immunogenicity of GPE.

Unlike thyrotrophin-releasing hormone (TRH) which has an uncommon pyro-glutamate moiety which is ideal for generating antibodies, small molecules like GPE often lack such suitable antigenic sites. This makes it very difficult to generate suitable antibodies for use in routine radioimmunoassay (RIA). There have been a number of attempts to measure by radioimmunoassay small peptides such as the metabolite of cholecystokinin, Gly-Trp-Met, using a variety of methods. However, many of these methods often require complex sample preparation, or are difficult to measure accurately in samples because of cross reactivity with similar small molecules. Consequently in order to measure such small molecules accurately researchers have been forced to rely on expensive and time consuming alternative methods such as gas chromatography/mass spectroscopy (GC/MS) or liquid chromatography/mass spectroscopy (LC/MS).

For nearly 30 years N-[(4-hydroxyhydrocinnamoyl)oxy]succinimide (Bolton and Hunter reagent) has been routinely used to label multiple proteins and peptides lacking a suitable iodination site to a high specific activity. In the original paper, the authors noted that the affinity of the antibody for the ligand was influenced by the method used to radiolabel the antigen.

The affinity of the antibody for the hapten is often modified by the cross-linking agent used because the coupling procedure results in a loss of charge for the hapten (12, 14). As a result there have been a number coupling schemes devised to minimise the effect of the linker. For small peptides, however, there is often a lack of suitable alternatives. The coupling of the Bolton and Hunter reagent to free amino groups can result in the net loss of charge for the molecule. Bolton and Hunter reagent may increase the antibody's affinity for an antigen if the coupling technique employed resulted in a loss of charge. If this were the case then the additional conversion of assay standards and samples to the Bolton and Hunter derivative would most likely increase the sensitivity of an assay for that molecule.

We therefore derivatized plasma samples containing GPE with Bolton and Hunter reagent prior to processing by a routine homologous RIA. Using this assay we then went on to characterise the pharmacokinetics of GPE following i.v. administration.

The current findings constitute the first description of a new method for the measurement by radioimmunoassay of small compounds with poor immunogenicity. An additional aim was to use the new assay to establish the pharmacokinetic properties of GPE, a small peptide that has been shown to have marked neuroprotective effects and a potential clinical application (2, 3, 5).

Iodination is one of the most common means of radio-labelling peptides to a high specific activity, but requires the presence of a tyrosine residue (21, 22). The Bolton and Hunter reagent has been routinely used to label multiple proteins and peptides that lack a suitable iodination site to high specific activity for over 30 years. Other compounds have also been used, including Assoian reagent and N-ethylmaleimide, but these often have limited or undesired cross-reactivity, which significantly alters the antigenicity of peptides and therefore limits their ability as a useful radiolabeling reagent (6, 8, 10, 14). The Bolton and Hunter reagent has also been used to minimise the effect of iodination of any tyrosine moieties as these have a relativity high antigenic propensity and are likely to be on or near an antibody recognition site (12, 23). Consequently iodination of tyrosine residues may disrupt the site of binding and may result in reduced affinity for the tracer.

In their original paper, Bolton and Hunter noted that antisera to peptides are generally selected for their reactivity with antigens iodinated by the radiolabeling protocol employed. Furthermore the linker used in coupling the hapten to the carrier protein can also influence the specificity and sensitivity of the antibody. A classic example is antibodies to TRH, which have been generated using TRH coupled by various mechanisms to carrier molecules. Often TRH coupled to the carrier has a higher affinity for the antibody than for TRH alone (7, 24). Generally this is because the coupling technique results in a loss of charge of the hapten, significantly altering its antigenicity.

A further example is t-Boc iodotyrosine n-hydroxysuccinimide derivatives of peptides that result in a 25% higher antibody titre than when using the unblocked peptide. This is consistent with the method used for conjugating the hapten to albumin protein. As a result a number of alternate coupling schemes have been devised that can reduce the influence of the linker usually by linking the hapten to the carrier at a site away from the potential antigenic site. For small peptides and compounds, however, there is often a lack of suitable alternatives.

Although the precise mechanisms that underlie our findings are not known with certainty, one theory is that in the studies presented here, the coupling of Bolton and Hunter reagent to an amino group of GPE may result in a loss of charge identical to that of the hapten-carrier complex. We could therefore take advantage of this loss of charge during the derivatization to identify antibodies of a higher specific activity rather than trying to identify antibodies against the underivatized compound of interest alone. The difference in charge between YGPE (which has a charged amino group) and BH-GPE (which is structurally similar having an identical phenol ring but lacking the charged amino group) may be a reason for the 4000 fold increase in affinity for the CK5 antibody. Another advantage of Bolton and Hunter reagent is that it is structurally similar to tyrosine lacking only the amino group. Therefore it could be possible to increase the size of the hapten by addition of a tyrosine group to the N terminus (or any available amino group), which would increase the complexity of the hapten and hence its immunogenicity. An additional advantage of this technique is that underivatized GPE does not need to be removed from the iodination mix because of its inability to displace BH-GPE.

The use of Bolton and Hunter reagent as a sample derivatization agent is suitable for one or more of a number of reasons. Other labelling techniques typically rely on complex extractions and derivatization steps. Here we have developed a simple two step procedure prior to the RIA. In addition the simple derivatization chemistry and low price enables it to be used in excess during the derivatization of samples in a manner commonly used in the derivatization of samples prior to HPLC. In addition the extraction step used was also compatible with HPLC analysis of peptides and metabolites of GPE and we have used this to our advantage by using the same sample preparation for both the HPLC and RIA analysis. Derivatization of the sample also means that a more robust homologous assay system could be developed which uses a single concentration of tracer, consumes less of it and therefore is more practical when tracers are expensive or difficult to synthesis.

In addition to Bolton-Hunter reagent, other materials can be useful for derivatizing GPE. They include a variety of iodination and cross-linking reagents.

A. Cross-linking Iodinatable Reagents Reactive Toward Amines, Carbohydrates, Carboxyls or Nonselective (Photoreactive)

Reagents that cross link certain moieties on a molecule include N-Hydroxysuccinimidyl-4-azidosalicylic acid (NHS-ASA) or Boc-L-tyrosinehydroxysuccinamide ester.

B. Reagents Used in Iodination of Proteins

Reagents used for iodination of proteins include N-Succinimidyl-3-(4-hydroxyphenyl)propionate (Bolton-Hunter reagent (SHPP); Sulfosuccinimidyl-3-(4-hydroxyphenyl)propionate (Water soluble Bolton-Hunter reagent (Sulfo-SHPP); 3-(4-Hydroxyphenyl)propionic acid hydrazide) (HPPH); Iodination Reagent (Pierce).

### EXAMPLES

The following examples are intended to illustrate embodiments of this invention, and are not intended to limit the scope of the invention. Others of skill in the art can use the teachings and descriptions herein to arrive at obvious variations of these embodiments and all such variations are considered to be within the scope of this invention.

### Example 1

### Materials and Reagents Used For Producing GPE Antisera

Gly-Pro-Glu (GPE; SEQ ID NO:1) and the di-peptides Gly-Pro and Pro-Glu were purchased from Bachem AG (Basal, Switzerland). The two artificial peptides designed to consist of the tripeptide GPE extended at the N-terminus so as to contain either a tyrosine residue for iodination (Tyr-Gly-Pro-Glu; YGPE; SEQ ID NO:2) and a lysine for conjugation (Lys-Tyr-Phe-Gly-Gly-Pro-Glu; KYFGGPE; SEQ ID NO:3) and were purchased from Macromolecular Resources, Fort Collins, Colo., USA. Sodium ¹²⁵iodide was purchased from Amersham Biosciences UK Ltd, Buckinghamshire, UK. Bovine serum albumin (BSA) was supplied by Roche Products New Zealand Ltd, Auckland, New Zealand. 6-aminoquinolyl-N-hydroxysuccinimidyl Carbamate (AccQfluor™) reagent was purchased from Waters Corporation, Milford Mass. USA. All remaining chemicals unless indicated were purchased from Sigma-Aldrich Pty. Ltd., Sydney, Australia.

All animal experimental protocols were approved by the University of Auckland Animal Ethics Committee.

### Example 2

### Preparation of Immunogen

All animal experimental protocols were approved by the University of Auckland Animal Ethics Committee.

GPE (1.5 mg) and KYFGGPE (1.5 mg) peptide were conjugated via their N-termini to KLH using glutaraldehyde. The peptides were conjugated to KLH via their N-terminus so that antibodies would be generated towards the C-terminus of the peptide and therefore minimizing potential cross reactivity with IGF-1.

### Example 3

### Production of Antisera Against GPE I

Three New Zealand White rabbits were injected subcutaneously with 200-300 μg of a peptide-conjugate immunogen (a 1:1 mixture of GPE conjugated to keyhole limpet hemocyanin (KLH) using glutaraldehyde (GA) and KYFGGPE conjugated to KLH using GA) emulsified in Freund's complete adjuvant (primary immunization). Booster injections with the same immunogen emulsified in Freund's incomplete adjuvant were given at 3 to 4 weekly intervals. Blood samples were taken from the marginal ear vein 10 days after each injection for titer determination, and booster immunizations continued until a suitable titer was achieved (9 injections). The rabbits were then anesthetized and killed by terminal exsanguination. The blood was allowed to clot, then centrifuged, and the supernatant serum recovered. This serum contains the polyclonal anti-GPE antibody, which we refer to as CK5 antibody, and was frozen at −20° C. until ready for use. Since the presence of other non-GPE related immunologic reactions does not interfere with the reaction between GPE and its antibody (anti-GPE antiserum), the polyclonal CK5 antibody did not undergo any further purification. Characterization of the CK5 antibody was performed using both double antibody radioimmunoassay and immunohistochemical techniques.

### Example 4

### Production of Antisera Against GPE II

The combined peptide-conjugates (250 μg) were then emulsified in Freund's complete adjuvant for the primary immunization and injected subcutaneously into six New Zealand white rabbits. For booster injections the combined peptide-conjugates were emulsified in Freund's incomplete adjuvant and were given at 3 to 4 weekly intervals. Blood samples were taken from the marginal ear vein 10 days after each injection for antibody titre determination. Immunizations continued until a suitable antibody titre was achieved.

### Example 5

### Antibody Characterization I

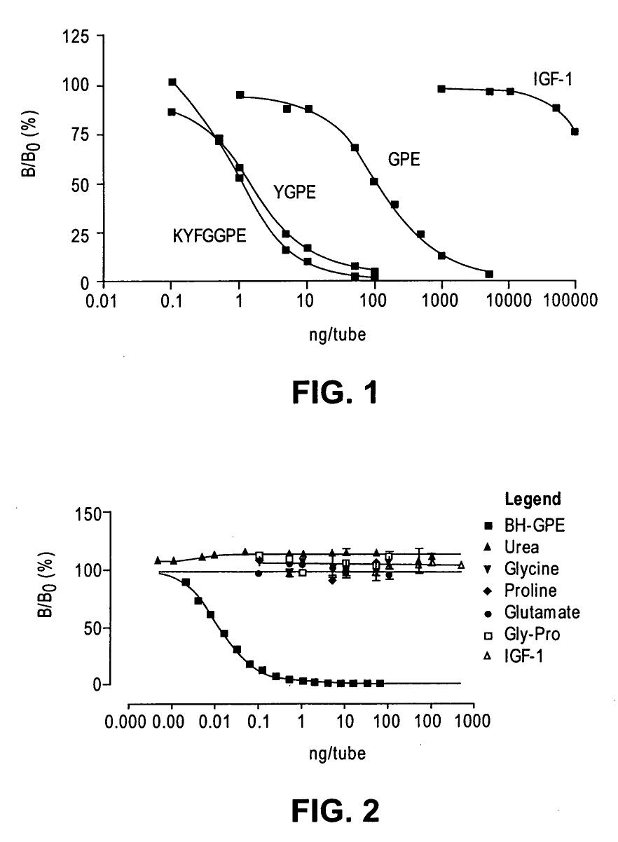

The CK5 antibody was characterized using a double antibody radioimmunoassay technique. Tubes containing either 100 μL 0.02 M phosphate buffered saline assay buffer or peptide (GPE, YGPE, KYFGGPE, or IGF-1) dissolved in assay buffer at various concentrations were pre-incubated with CK5 antibody (diluted at 1:600) for 24 h at 4° C. I¹²⁵-labeled YGPE (10,000 cpm) was then added to the tubes. After a further 48 h incubation at 4° C., the bound and free GPE were separated by adding donkey anti-rabbit serum (1:100). The tubes were incubated with this second antibody overnight at 4° C. before centrifugation (3,200 rpm for 30 min), after which the supernatant was aspirated, and the precipitate counted in a gamma counter. The results are shown in FIG. 1. One antibody, CK5, was identified. Under the assay conditions described above, CK5 exhibits 14.7% specific binding to GPE at a final titer of 1:600, using I¹²⁵-labeled YGPE as the tracer. Unlabeled GPE was able to displace I¹²⁵-labeled YGPE with an ED₅₀ of approximately 200 ng/tube. Importantly, the CK5 antibody does not cross-react significantly with the GPE parent molecule, IGF-1. The specificity of the CK5 antibody was further confirmed by Western blot and dot blot analysis. GPE immunoreactivity was detected when membranes were incubated with CK5 overnight at 4° C. whereas pre-absorption of CK5 overnight with excess unlabeled GPE completely abolished GPE immunoreactivity. Thus, CK5 is a specific antibody that recognizes and competitively binds GPE.

### Example 6

### Antibody Characterization II

GPE were characterized using a standard double antibody radioimmunoassay technique modified from Example 5 above. Briefly, tubes containing an antibody at a dilution of 1:600, 1:6000 and 1:60000 were titred against 15000 cpm/tube of ¹²⁵I-YGPE or ¹²⁵I-BH-GPE in assay buffer (0.05 M phosphate buffered saline containing 0.2% BSA and 0.1% Triton X-100, pH 7.8) for 24 h at 4° C. Pre-precipitated second antibody complex (sheep anti-rabbit γ-globulin in 0.01 M phosphate buffered saline (PBS) containing 8% PEG-6000 and 0.1% normal rabbit serum, 1 ml/tube) was then added and the tubes incubated for 2 h at room temperature. Samples were then centrifuged at 3000×g for 45 min at 4° C., the supernatant decanted and the precipitate counted using a gamma counter (Cobra I, Packard Bioscience).

### Example 7

### Preparation of Tracers

GPE conjugated to Bolton and Hunter reagent (BH-GPE) was synthesized as follows.

GPE (358 μg) was resuspended in 200 μl 0.1 M phosphate buffer pH 8.0. Twenty μl of 20 mM Bolton and Hunter reagent in dimethyl sulfoxide (DMSO) was then added, the reaction vortexed immediately and incubated overnight at 4° C. Five μl of the reaction was then diluted to 200 μl with 0.01 M PBS pH 7.4 and 20 μl removed for iodination. The BH-GPE and Tyr-Gly-Pro-Glu (YGPE) were iodinated using the chloramine T method (15). Iodinated tracer and unincorporated GPE were separated from free iodine using a G10 Sephadex gel filtration column and collected into 45×4 min fractions. The peak fractions were pooled and used for subsequent analysis.

### Example 8

### Detection of Bolton and Hunter Derivatized GPE Using the CK5 Antibody

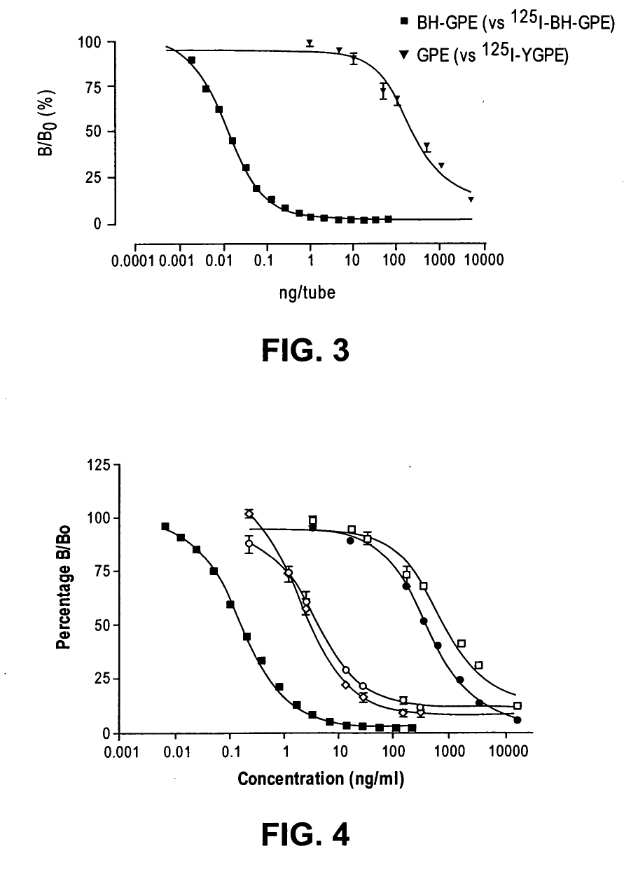

The CK5 antibody was prepared as described in Examples 1-4, and characterized using a modified double antibody radioimmunoassay technique. The CK5 antibody was used at a final dilution of 1:18,000. Tubes containing either 100 μL assay buffer (pH 7.8, 0.05 M sodium phosphate), peptide (GPE, glycine, proline, glutamic acid, GP, PE, or IGF-1), or urea were derivatized with Bolton and Hunter reagent, N-succinimidyl-3-[4-hydroxyphenyl] propionate, and dissolved in assay buffer. The tubes were incubated with CK5 antibody and I¹²⁵-labeled Bolton and Hunter derivatized GPE (15,000 cpm) for 48 h at 4° C. The bound and free GPE were then separated by adding sheep anti-rabbit gamma globulin (1:100). The tubes were incubated with this second antibody for 4 h at room temperature before centrifugation (3,200 rpm for 45 min), after which the supernatant was tipped off and the precipitate counted in a gamma counter. The results are shown in FIGS. 2 and 3. Under the assay conditions described above, CK5 exhibits 50% specific binding to Bolton and Hunter derivatized GPE at a final titer of 1:18,000, using I¹²⁵ labeled Bolton and Hunter derivatized GPE as the tracer. Unlabeled Bolton and Hunter derivatized GPE was able to displace I¹²⁵ labeled Bolton and Hunter derivatized GPE with an ED₅₀ of approximately 0.01 ng/tube and the minimal detectable level of GPE was 0.005 ng/tube. The addition of either rat or human plasma to the standard curve resulted in parallel displacement. Importantly, CK5 antibody does not cross-react significantly with Bolton and Hunter derivatized glycine, proline, glutamic acid, GP, PE, IGF-1, or urea. The assay of differing volumes of rat plasma (25, 50, 75, 100 μL) containing known amounts of added GPE resulted in a linear relationship.

The specificity of CK5 was further confirmed by Western blot and dot blot analysis. GPE immunoreactivity was detected when membranes were incubated with CK5 overnight at 4° C.; whereas preabsorption of CK5 overnight with excess unlabeled GPE completely abolished GPE immunoreactivity. Thus CK5 is a specific antibody that recognizes and competitively binds Bolton and Hunter derivatized GPE with a higher ED₅₀ than for underivatized GPE. The ED₅₀ for modified displacement of Bolton and Hunter derivatized GPE was 0.0094 ng/tube (antibody diluted at a final dilution of 1/18,000), whereas the ED₅₀ for the standard displacement was 199.8 ng/tube (antibody diluted at a final dilution of 1/600).

### Example 9

### Sample Extraction

Fifty μl of plasma was added to 400 μl of 0.4 M H₂SO₄, vortexed and incubated on ice for 5 min. Fifty μl of 10% sodium tungstate was added and the samples were incubated for 20 min on ice, vortexing at 0 and 10 min before centrifugation at 20,000×g for 20 min at 4° C. The supernatant (450 μl) was then transferred to a new tube and stored at −80° C. IGF-1 is proteolytically cleaved by a acid protease present in serum to des IGF-1 and GPE. Therefore in order to minimize the levels of endogenous GPE, the control pool plasma stocks were prepared from starved dwarf rats which have negligible IGF-1 levels.

### Example 10

### Sample Derivatization for Radioimmunoassay

Samples (450 μl) or assay standards were added to 450 μl 0.1 M phosphate buffer (pH 8.0) and vortexed. Ninety μl of a 20 mM solution of Bolton and Hunter reagent in DMSO was added and the samples immediately vortexed and incubated for 4 h at room temperature. Derivatized samples were then dried down overnight before reconstitution in 450 μl assay buffer at the time of assay.

### Example 11

### Radioimmunoassay Procedure

Primary antibody (CK5) was diluted in assay buffer to an initial concentration of 1:6000. One hundred μl of sample, control or standard (0.001-64 ng/ml BH-GPE) were then incubated with 100 μl primary antibody and 100 μl ¹²⁵I-BH-GPE at 15000 cpm/tube for 72 h. One ml of the pre-precipitated second antibody complex (sheep anti-rabbit γ-globulin in 0.01 M PBS containing 8% PEG 6000 and 0.1% normal rabbit serum) was then added and the tubes incubated for 2 h at room temperature. Samples were then centrifuged at 3000×g for 45 min at 4° C., before the supernatant was decanted and the precipitate counted using a gamma counter. Results were expressed as a percent of displacement of bound ¹²⁵I tracer and the IC₅₀ determined.

### Example 12

### HPLC and Separation of GPE

GPE standard in PBS or a sample of plasma spiked with 25 ng/ml GPE were resolved and eluted using a 1 ml/min mobile phase of 3% acetonitrile with 0.025% trifluoroacetic acid in water on a Aqua 5 μ250×4.6 mm C18 column (Phenomenex, Auckland, New Zealand) connected to a Waters 2695 Alliance separation module and Waters 2996 PDA detector with an absorbance set at 200 nm. 30×1 min fractions were collected, dried down and resuspended in 450 μl 0.1 M phosphate buffer (pH 8.0).

### Example 13

### Sample Derivatization for HPLC

The metabolism of GPE in plasma was assessed by HPLC using a modified AccQfluor method (17). Briefly, samples were derivatized with AccQfluor reagent which converts primary and secondary amino groups to fluorescent derivatives, resolved by reverse phase HPLC, and compared to known amino acid and GPE standards.

The reversed phase HPLC system consisted of a Waters 2690 Alliance separation module, a 300×3.9 mm C18 Pico-tag (Waters) column at 37° C. and a Waters 474 fluorescence detector set at excitation and emission wavelengths of 250 and 395 nm respectively. This was linked to a PC running the Waters Millennium³² program (Waters Corporation, Milford Mass. USA). The mobile phase consisted of a complex gradient with acetonitrile from 0 to 16%, buffer made up with 80 mM sodium acetate, 3 mM triethylamine, 2.7 mM EDTA, brought to pH 6.43 with orthophosphoric acid and was run over 112 min, at a flow rate 1.2 ml/min at 37° C.

### Example 14

### Sensitivity of Antiserum

One Rabbit (CK5) generated a sufficiently high antibody titre to YGPE and BH-GPE. The antiserum had a 50% maximum binding at a final concentration of 1/900 for ¹²⁵I-YGPE. In contrast ¹²⁵I-BH-GPE had 50% binding at a final concentration of 1/18000.

When using underivatized GPE the IC₅₀ was 665.7 and 408 ng/ml for ¹²⁵1-YGPE and ¹²⁵I-BH-GPE tracers, respectively (FIG. 4). In contrast, the IC₅₀ for GPE derivatized with Bolton and Hunter reagent was 0.175 ng/ml when using ¹²⁵I-BH-GPE tracer, a 4000 fold increase in sensitivity. In addition the lowest detectable level of GPE was 7 pg/ml at the 95% confidence limit. Because of the increased sensitivity and higher titre when using ¹²⁵I-BH-GPE as the tracer, all subsequent analysis was performed using ¹²⁵I-BH-GPE as the tracer.

### Example 15

### Specificity

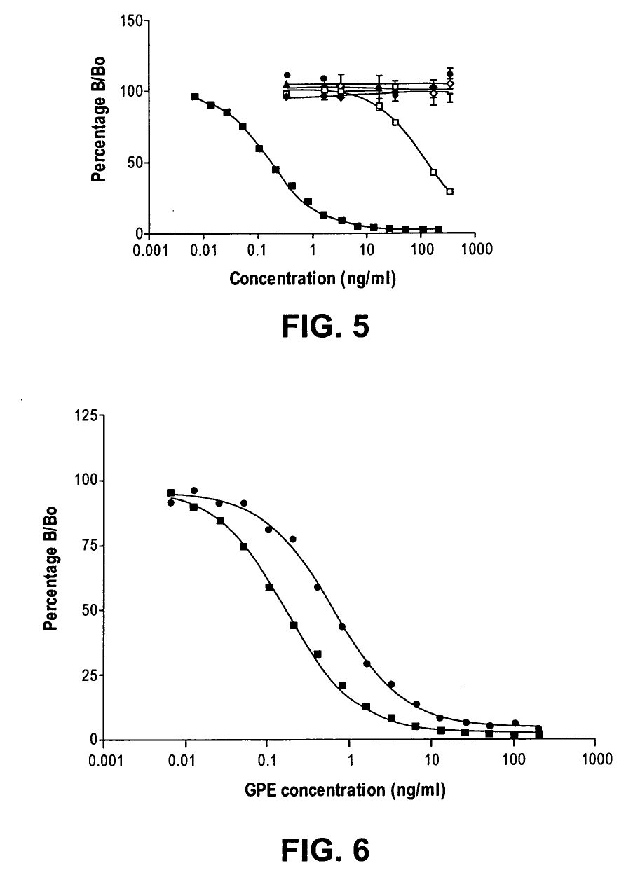

In order to investigate the assay specificity, BH-GPE was compared against both normal and Bolton and Hunter derivatized forms of Glycine, Proline, Glutamate, Gly-Pro and Pro-Glu (FIG. 5). Derivatized Pro-Glu had some minor (0.17%) cross-reactivity (IC₅₀ 101.4 ng/ml), however, no cross-reactivity of the CK5 antisera (see example 11—rabbit anti-GPE antiserum) against either the underivatized or the Bolton and Hunter derivatized forms of Glycine, Proline, Glutamate and Gly-Pro were observed (FIG. 2) nor with Bolton and Hunter derivatized forms of TRH or urea (data not shown).

### Example 16

### Recovery and Efficiency

In preliminary experiments GPE incubated in plasma samples for 30 min at 37° C. indicated a rapid loss of GPE due to proteases present in the plasma whereas samples incubated with a protease inhibitor cocktail described above had no loss of GPE. Therefore the general protease inhibitor cocktail was added to all samples and recovery standards to prevent loss of GPE prior to extraction. In addition there are two further steps where a significant loss of peptide can occur. The first loss is during the extraction procedure and the second is in the derivatization step. Using plasma spiked with ³H-GPE (SibTech Inc. Newington, Conn., USA) the extraction recovery was 83±3% (mean±SD of 6 experiments). Since there is also the potential for loss of GPE due to the derivatization step, a study was set up investigating different conditions of Bolton and Hunter derivatization of GPE. Altering the concentration or type of buffer used for derivatization (0.1 and 0.01 M phosphate or borate buffer) did not alter the IC₅₀. Similarly, there was no change in IC₅₀ values between the 4 hour incubation at room temperature and an overnight incubation at 4° C. We also found that at stock concentrations above 20 mM the Bolton and Hunter reagent precipitated out of solution when mixing the DMSO and Phosphate buffer. The results suggested that 20 mM Bolton and Hunter reagent and a derivatization time of 4 h was sufficient to achieve 100% derivatization.

### Example 17

### Parallel Displacement

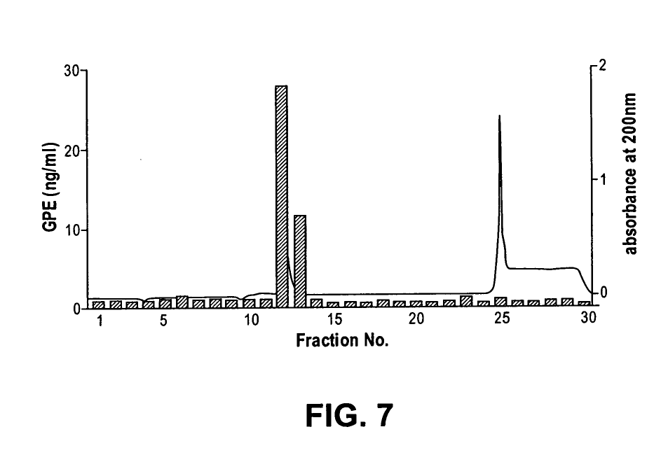

In order to identify if other unidentified small peptides in plasma are cross-reacting with the CK5 antiserum GPE (concentration range 0.006-213 ng/ml) was measured in 100 μl extracted plasma ‘spiked’ with of GPE or in normal buffer (FIG. 6). GPE in plasma had a small change of IC₅₀ from 0.175 to 0.652 ng/ml, but was otherwise parallel indicating there were no cross-reactive compounds in plasma. In addition, plasma spiked with 25 ng/ml GPE was first separated using HPLC and the fractions collected. RIA analysis of the fractions identified a single immunoreactive peak that corresponded to the elution position of GPE standard. This shows that the only immunoreactive compound present in plasma that is detected by the CK5 antibody is GPE (FIG. 7).

### Example 18

### Precision

Inter-assay and intra assay coefficient of variation (CV) was calculated using GPE spiked starved dwarf rats plasma control pools from six replicates of 5, 10 and 25 ng/ml standards over 10 different experiments over a 5 week period. The observed intra-assay CV was 7.4±2.9, 6.4±2.8 and 10.9±5.0 percent for 5, 10 and 25 ng/ml respectively. The inter-assay mean concentration (ng/ml±SE) was 7.2±0.2, 13.2±0.4 and 30.6±1.0 for 5, 10 and 25 ng/ml and suggest a basal level of approximately 2.5 ng/ml in starved dwarf rats. The intra-assay CV values were 11.0, 9.1 and 10.2% respectively. All the CV values were below 15%, which is within the accepted standard FDA guidelines for precision testing (19, 20).

### Example 19

### Radioimmunoassay (RIA) Measurement of GPE in Biological Fluids and Tissues Using CK5 Antibodies and Bolton and Hunter Derivatized GPE

This procedure uses a pre-RIA derivatization of the GPE-containing sample and comprises three steps: initial preparation of the sample using a tungstate extraction procedure to remove large proteins and to prevent overloading of the Bolton and Hunter reagent with an excess of amino groups; derivatization of samples and standards with Bolton and Hunter reagent; and a standard RIA protocol combining the CK5 antibody, ¹²⁵I-labeled Bolton and Hunter derivatized GPE as the tracer, and PEG precipitation.

Acid Tungstate Precipitation From Blood, CSF and Urine

Whole blood was collected into collection tubes containing a metalloprotease inhibitor, for example Sigma protease inhibitor cocktail, and centrifuged at 3,000 g for 15 min at 4° C. The supernatant (plasma) was transferred into a new tube and stored at −80° C. until ready for assay. CSF and urine were collected into collection tubes containing a metalloprotease inhibitor, for example Sigma protease inhibitor cocktail, and stored at −80° C. until ready for assay. The samples were thawed on ice. During-thawing of the samples, 800 μL of 0.04 M sulfuric acid was added to 1.5 mL micro-centrifuge tubes and incubated on ice. Aliquots (100 μL) of the samples were transferred to the micro-centrifuge tubes, and the tubes were vortexed and incubated on ice for 5 min, after which 100 μL of 10% sodium tungstate was added. The tubes were vortexed and incubated on ice for 10 min, twice. The tubes were then centrifuged at 20,000 g for 20 min at 4° C., after which 900 μL of the acid tungstate-treated sample was removed to a new micro-centrifuge tube and stored at −80° C. Tritiated GPE was used to determine a recovery level of 90-92% for the extraction procedure.

Acid Tungstate Precipitation From Tissue

All steps were performed on ice to prevent degradation of GPE. Approximately 50 mg of tissue was accurately weighed in a micro-centrifuge tube and 5 μL of protease inhibitor and 160 μL of 0.67 N H₂SO₄ added. The sample was homogenized for 3 min with a micro-centrifuge tube fitting pestle (approximately 100 strokes) or until a liquid homogenate was obtained. The pestle was rinsed into the tube with 400 μL of water using a pipette, and the tube sonicated for 1-5 sec. The probe was rinsed with 180 μL of water and the rinse added to the homogenate, then 60 μL of 10% sodium tungstate added, and the tube vortexed and incubated on ice for 10 min, twice. The tube was then centrifuged at 20,000 g for 20 min at 4° C. and the supernatant transferred to a new tube. To the pellet was added 100 μL of water, and the pellet was resuspended by vortexing and sonication for 1-5 sec, again rinsing the probe with 100 μL of water. The pellet was then centrifuged at 20,000 g for 20 min at 4° C., and the supernatant added to the original supernatant. Chloroform (100 μL) was added, and the tube was vortexed and centrifuged at 20,000 g for 5 min at 4° C. One mL of the upper layer was transferred to a new tube, taking care not to disturb the chloroform layer, and frozen at −80° C. Tritiated GPE was used to determine a recovery level of 92-94% for the extraction procedure.

Bolton and Hunter Derivatization of Samples

To 100 μL of thawed treated sample was added 100 μL of 0.1 M phosphate buffer, and the mixture vortexed, after which 20 μL of 20 mM Bolton and Hunter reagent was added, and the samples incubated at room temperature for 4 h. The derivatized samples were then lyophilized overnight, re-suspended in 100 μL of assay buffer, and transferred to polypropylene plastic assay tubes (12×75 mm). For the standards, 100 μL of Bolton and Hunter reagent was added to 1 mL of standard sub-stock containing 640 ng/mL phosphate buffer. The standard was incubated at room temperature for 4 h. The derivatized standards were lyophilized overnight, and re-suspended in 1 mL assay buffer.

Radioimmunoassay of Bolton and Hunter Derivatized GPE II

Rabbit CK5 antibody was used at a final dilution of 1:18,000 in assay buffer, and ¹²⁵I-labeled Bolton and Hunter derivatized GPE (BH-GPE) was used as tracer at 150,000 cpm/mL in assay buffer. Sheep anti-rabbit gamma globulin (1% in 0.01 M PBS with 8% PEG), with 0.05% normal rabbit serum, incubated for 90 min at 4° C. before use, was the second antibody precipitation reagent.

BH-GPE sub-stock containing 640 ng/mL BH-GPE was serially diluted to concentrations ranging from 640 ng/mL to 0.0002 ng/mL. Three 100 μL aliquots of each concentration were then transferred to polypropylene plastic assay tubes (12.times.75 mm). To each sample was added 100 μL antibody and 100 μL tracer, and the tubes vortexed. The samples were incubated for 72 h at 4° C., and 1 mL of secondary antibody reagent was added. The sample was incubated at room temperature for 2 h, then centrifuged at 3,000 g for 45 min at 4° C. The supernatant was poured off and counted for 1 min in a Cobra Gamma counter (Packard Biosciences).

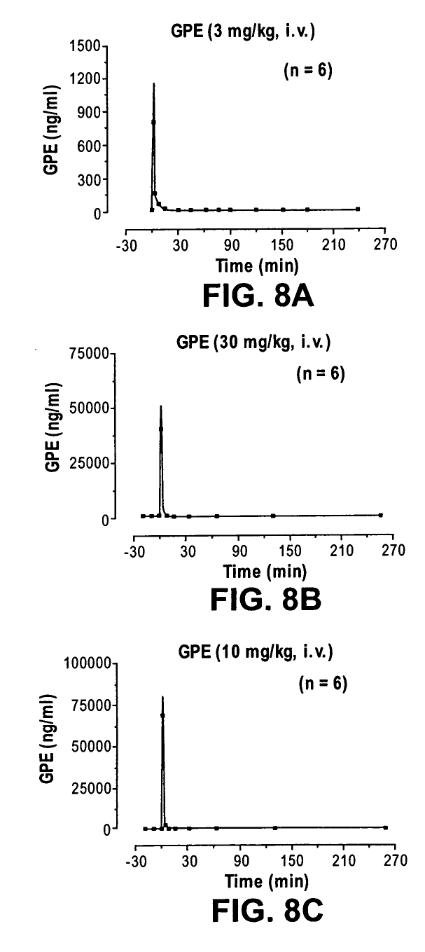

Injection of GPE intravenously into rats resulted in a rapid rise of GPE concentration followed by a rapid fall in the amounts of GPE recovered. The results are shown in FIGS. 8A-8C. FIG. 8A shows the results of injecting 3 mg/kg., i.v., FIG. 8B shows the results of injecting 30 mg/kg/i.v., and FIG. 8C shows results of injecting 100 mg/kg, i.v. The addition of the CK5 antibody and ¹²⁵I-Bolton and Hunter derivatized GPE tracer in a radioimmunoassay allows the specific measurement of GPE plasma concentrations in samples following intravenous dosing. Using the CK5 antibody, GPE is detectable in blood following dosing and has a half-life of approximately 1-2 min.

### Example 20

### Reverse HPLC Using AccQTag® Derivatization

GPE-containing samples were prepared as in Example 3 immediately above. The samples were derivatized by the Waters AccQTag® method, which involves incubation of the sample with 10 mM 6-aminoquinolyl-N-hydroxysuccinimidyl carbamate in acetonitrile and borate buffer at 55° C. for 10 min and converts primary and secondary amino groups to fluorescent derivatives, before being transferred to the HPLC injection vial. These reaction products were resolved by HPLC and compared to known amino acid standards. The reverse phase HPLC system consisted of a Waters 2690 Alliance separation module, a 300×3.9 mm C18 Pico-tag (Waters) column at 37° C., and a Waters 474 fluorescene detector set at 250 nm excitation, 395 nm detection, gain 100. This was linked to a PC running the Waters Millennium³² program (Waters Corporation, Milford, Mass. 01757). The mobile phase consisted of three components: component A was MilliQ water, component B was a buffer made up with 80 mM sodium acetate, 3 mM triethylamine, 2.7 μM EDTA, brought to pH 6.43 with orthophosporic acid, and component C was acetonitrile. The mobile phase was run in the gradient shown in Table 1 below over 112.1 min, at a flow rate of 1.2 mL/min at 37° C.

TABLE 1

| Time min) | % A | % B | % C | Curve |
|---|---|---|---|---|
| 0 | 49.9 | 49.9 | 0.2 | 6 |
| 13 | 48.7 | 48.7 | 2.6 | 6 |
| 27 | 48.6 | 48.6 | 2.8 | 6 |
| 50 | 48.5 | 48.5 | 3 | 6 |
| 75 | 46 | 46 | 8 | 6 |
| 82 | 45 | 45 | 10 | 6 |
| 98 | 43 | 43 | 14 | 6 |
| 108 | 41.5 | 41.5 | 17 | 11 |
| 108.1 | 40 | 0 | 60 | 11 |
| 112.1 | 49.9 | 49.9 | 0.2 | |

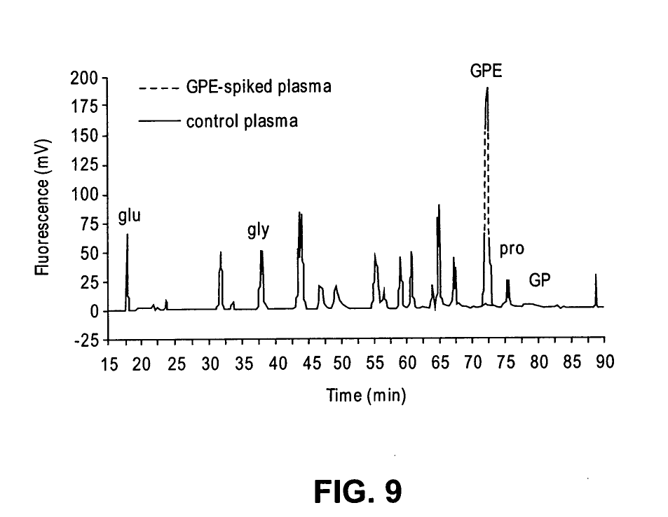

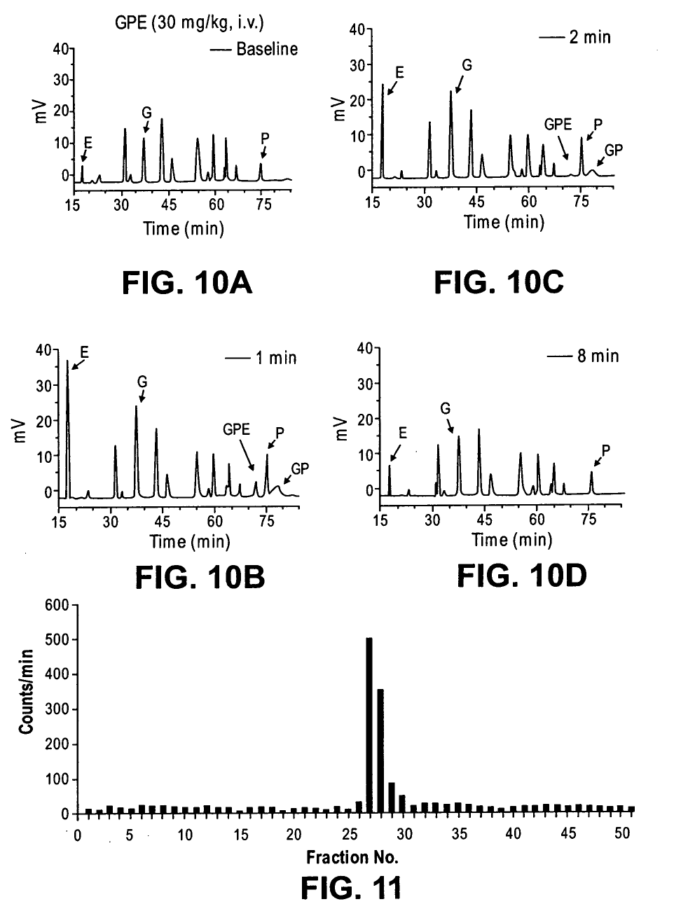

The results are shown in FIGS. 9 and 10. The rpHPLC elution profile shows that GPE elutes with a retention time of approximately 72 min, and the GPE peak is sharp and resolved and clearly detectable above control plasma. No GPE was detected in ‘unspiked’ control plasma. This method has also been repeated with tritiated GPE, which eluted with the same retention time.

FIGS. 10A-10D depict results of HPLC studies in which GPE was measured at different times after intravenous injection (30 mg/kg, i.v.). FIG. 10A is of a baseline study in which no GPE was administered. FIG. 10B shows the results 1 minute after GPE injection. There is a peak at about 72 minutes, corresponding to GPE, and the peaks at about 17 min, 37 minutes and at about 76 minutes are larger than in FIG. 10A, reflecting the increase in glutamate (E), glycine (G) and proline (P), respectively, in the sample. FIG. 10C shows the results 2 minutes after GPE injection. Compared to FIG. 10B, the peaks for GPE, E and P and G are reduced FIG. 10D shows the results 8 minutes after injecting GPE. The peak for GPE is gone, demonstrating a rapid degradation of GPE in the circulation. Reverse phase HPLC of AccQTag derivatized GPE-containing samples is a reliable and sensitive method to detect GPE.

### Example 21

### Reverse Phase HPLC Using A Hypercarb® Column

GPE-containing samples were prepared as in Parts 1a and 1b of Example 3. Samples were thawed on ice before being transferred to the HPLC injection vial. The reverse phase HPLC system consisted of a 100×4.6 mm Hypercarb 5 μm (Hypersil) column between a Waters Wisp Autosampler (Waters) and a BioCAD Sprint workstation (Applied Biosystems) running Version 2.062 of the BioCAD workstation software and an Advantec fraction collector set to collect 0.5 mL fractions. Samples were run onto the column in a mobile phase consisting of 10% methanol, 0.1% trifluoroacetic acid in MilliQ water, then eluted using a linear gradient with a mobile phase consisting of 90% methanol, 0.1% trifluoroacetic acid in MilliQ water using 0-100% gradient over 25 min as in Table 2 below, with a flow rate of 1.0 mL/min at room temperature. UV absorbance detection was set at 220 nm, and 0.5 mL fractions were collected into 5 mL scintillation vials from time of injection until the end of the gradient. Scintillation fluid (4 mL) was then added to each vial, and the samples counted in a 14XX Rack-beta scintillation counter (Wallac, Perkin Elmer). Results are shown below in Table 2 and in FIG. 11.

TABLE 2

| | 10% MeOH/ | 90% MeOH/ | |
|---|---|---|---|
| Time(min) | 0.1% TFA | 0.1% TFA | Event |
| 0 | 100 | 0 | |
| 5 | 100 | 0 | |
| 10 | 100 | 0 | Injection/fraction start |
| 35 | 0 | 100 | Fraction collection stop |
| 40 | 0 | 100 | |
| 41 | 100 | 0 | |
| 45 | 100 | 0 | |

The results are shown in FIG. 11. Tritiated GPE eluted in fractions 27 and 28. The GPE peak is sharp, resolved, and clearly detectable. Metabolic products of tritiated GPE (Gly-Pro and Proline) eluted in the void. The method was repeated with “cold” (non-tritiated) GPE and eluted with the same retention time.

### Example 22

### Polyclonal Antibody Production in Rabbits

Twelve female New Zealand White rabbits are injected subcutaneously with 600-1000 μg of peptide-conjugate emulsified in Freund's complete adjuvant. Three rabbits received a mixture of 300 μ of GPE conjugated to KLH using GA and 300 μg KYFGGPE conjugated to KLH using GA; three rabbits received a mixture of 300 μg GPE conjugated to KLH using GA and 600 μg GPE conjugated to KLH using diethyl carbodiimide, and six rabbits, primed with Bacillus Calmette-Guerin [BCG] vaccine, received 1000 μg CGPE conjugated to a purified protein derivative of tuberculin (Statens Serum Institut, Denmark) using sulfosuccinimidyl 4-(N-maleimidomethyl) cyclohexane 1-carboxylate (sulfo-SMCC, Pierce, Ill., USA). Booster injections emulsified in Freud's complete adjuvant were given at 3-4 weekly intervals. Blood samples were taken from the marginal ear vein 10 days after each injection for titer determination, and regular immunizations continued for up to 8 months (maximum 10 injections) until a suitable titer was achieved. Characterization of the anti-GPE antibody was performed using both the double antibody radioimmunoassay technique described herein.

### Example 23

### Passive Immunization Against GPE in Rats

Following hypoxic-ischemic injury, rats were treated with either GPE alone or GPE combined with anti-GPE antibodies. Nine pairs of adult Wistar rats (280-320 g) were prepared under halothane/O₂ anesthesia. The right side carotid artery was ligated. To facilitate the intracerebroventricular administration of treatment, a guide cannula was placed on the dura at stereotaxic coordinates AP+7.5 mm, R+1.5 mm. The rats were allowed to recover for 1 h and were then placed in an incubator with humidity 90±5% and temperature 31±0.5° C. for 1 h before hypoxia. The oxygen concentration was then reduced and maintained at 6±0.2% for 10 min. The rats were kept in the incubator for 2 h after hypoxia and then treated with either 3 μg GPE or 3 μg GPE plus 25 μL anti-GPE antibodies. A further 6 rats were used as normal controls. The rats were killed by being deeply anaesthetized with an overdose of pentobarbital and then transcardially perfused with normal saline followed by 10% buffered formalin. The brains were removed and kept in the same fixative for two days before being processed using a standard paraffin tissue procedure.

Coronal (8 μm) sections were cut from the striatum, cerebral cortex and hippocampus, mounted on glass slides and stained with Thionin and Acid Fuchsin. With the experimenter blinded to the treatment groups, the histological outcome was assessed using two levels: at the mid-level of the striatum and the level where the ventral horn of the hippocampus just appears. Dead neurons are acidophilic (red) and have contracted nuclei. An indirect technique was used to determine the extent of cortical damage; the area of intact cortical tissue in both hemispheres was measured using an image analyzer (SigmaScan (SPSS Science) Chicago, Ill.). Brain tissue with selective neuronal death and/or pan-necrosis was considered to be damaged. The right/left (R/L) ratio of area of intact cortex was compared between the treatment groups. Surviving neurons from both sides of the CA1-2 subregions of the hippocampus were counted from the boundary between CA3 and CA1-2 and towards CA1-2 for 600 μm. The R/L ratio of surviving neurons in the CA 1-2 subregions of the hippocampus was compared between treatment groups. Striatal damage was scored using the following scoring system: 0, no tissue damage; 1, <5% tissue damage; 2, <50% tissue damage; 3, >50% tissue damage. Passive immunization against GPE actively blocks the neuroprotective effects of GPE, suggesting that following GPE treatment, neuroprotective effects are specific to GPE action.

### Example 24

### Passive Immunization Against GPE in Lesioned Rats

Following a lesion with 6-hydroxy dopamine (6-OHDA), rats were treated with GPE either alone or combined with anti-GPE antibodies. Eighteen male Wistar rats (50-60 days; 280-310 g) were used for the study. Under 3% halothane anesthesia, the 6-OHDA (8 μg in 2 μL 0.9% saline containing 1% ascorbic acid) was administered into the right medial forebrain bundle (MFB) at stereotaxic coordinates AP+4.7 mm, R+1.6 mm, V−8 mm using a 100 μL Hamilton syringe with a 30 G needle controlled by a microdialysis infusion pump at an infusion rate of 0.2 μL/minute. The infusion needle was slowly withdrawn 5 minutes after the infusion. The surgery and procedures for the intracerebroventricular administration are described in Guan et al. (1993), The effects of IGF-I treatment after hypoxic-ischemic brain injury in adult rats, Journal of Cerebral Blood Flow and Metabolism 13:609-616. A 6 mm long, 21 G guide cannula is fixed on the top of the dura with coordinates of AP+7.5 mm, R+1.5 mm immediately after the injection of 6-OHDA. Either 3 μg GPE, 3 μg GPE plus 25 μL anti-GPE antibodies, or vehicle was infused into the right lateral ventricle 2 h after lesion at an infusion rate of 2 μL/min. Rats were then housed in a holding room with free access to food and water for the next two weeks. The rats were killed by being deeply anaesthetized with an overdose of pentobarbital and then transcardially perfused with normal saline followed by 10% buffered formalin. The brains were removed and kept in the same fixative for two days before being processed using a standard paraffin tissue procedure.

Coronal sections from the striatum and the substantia nigra compacta (SNc) were cut on a microtome to 8 μm thickness, mounted on chrome-alum coated slides, and air-dried. For staining, the sections were deparaffinized, rehydrated, washed with 0.1 M phosphate buffered saline (PBS), pretreated with 1% H₂O₂ for 20 min, washed with 0.1 M PBS (3×5 min), and incubated in rabbit polyclonal antisera raised against tyrosine hydroxylase (Protos Biotech, USA) diluted 1:500 with 1% goat serum for 48 h at 4° C. The sections were then washed in PBS (3×5 min) and incubated overnight at room temperature in donkey anti-rabbit biotinylated secondary antibody (1:200, Amersham Life Science). The sections were washed again in 0.1 M PBS, incubated in streptavidin-linked horse radish peroxidase (1:200, Amersham Life Science) for 3 h, washed again in PBS, and then treated with 0.05% 3,3′-diaminobenzidine tetrahydrochloride and 0.01% H₂O₂ to produce a brown reaction product. The sections were then dehydrated in a graded alcohol series, cleared in xylene, and coverslipped with mounting medium.

With the experimenter blinded to the treatment groups, the number of tyrosine hydroxylase-positive (TH-positive) neurons on both sides of the SNc are counted using light microscopic examination (20.times.magnification) at three representative levels (AP+4.2 mm, +3.8 mm and +3.4 mm). The average densities of TH staining on both sides of the SNc are measured using an image-analyser (Mocha image analysis software). The average density of TH staining in the striatum is also measured using three adjacent sections from the middle of the striatum. The average density from the background reading is also measured. The difference in average density between the background and TH staining is calculated and used for data analysis. Right/left (R/L) ratios of the number of TH-positive neurons and the R/L ratio of the average density of TH staining from each level of the SNc is compared between the two treatment groups using two-way ANOVA. The R/L ratio of the TH staining density from three striatal sections is averaged and compared between the two groups using the t-test. Data is presented as mean±SEM. The morphological changes in the SNc and the striatum are photographed using a Leitz Dialux light microscope (10× and 40× magnifications) or a digital camera and the images processed using Adobe Photoshop® and Pagemaker® software. Passive immunization against GPE actively blocks the neuroprotective effects of GPE, suggesting that following GPE treatment neuroprotective effects are specific to GPE action.

### Example 25

### Purification of the GPE Receptor

CK5 antibody is resuspended to a final concentration of 1/100 in 0.1M PBS pH 7.8. Sulfosuccinimidyl 2-[m-azido-o-nitrobenzamido]-ethyl-1,3-′-dithiopropionate (SAND) in DMSO is added to a final concentration of 10 mM, and the reaction mixture incubated in the dark at 37° C. for 30 min. Unreacted SAND is removed by dialysis against several changes of 0.1M PBS, pH 7.8. The CK5-SAND complex is then stored at −80° C. until used. Fresh frozen brain slices (60 μm thick) or cells grown in 80 cm cell culture dishes are briefly exposed to 100 μM GPE or vehicle in 0.1M PBS, excess unbound GPE is then washed off with three washes of PBS, and the samples are incubated in the dark for 1 h with CK5-SAND complex to enable the antibody to bind to the GPE, which is bound to its receptor. Photoactivation by 3-5 bright camera flashes results in crosslinking of the CK5-SAND complex to the GPE receptor. The cells/tissues are then solubilized in 1% Triton in 25 mM HEPES, pH 7.6; and CK5-SAND-receptor immunocomplexes are then purified using a HiTrap Protein G Column (Amersham Pharmacia Biotech) following the manufacturer's instructions. 2-Mercaptoethanol is then added to the sample extract to cleave the crosslinker; and the separated CK5 antibody and GPE receptor are resolved by two dimensional electrophoresis before blotting to PVDF membranes and staining with Coomassie blue.

GPE and vehicle treated extractions are compared and potential receptor bands identified. These bands are excised and sequenced using a gas-phase Sequencer (model 470A, Applied Biosystems) following the manufacturer's instructions or by MS/MS analysis.

We claim:

1. An antibody raised against a Bolton-Hunter (BH) reagent-derivatized-Gly-Pro-Glu (“GPE”).

2. The antibody of claim 1, wherein said Bolton-Hunter derivatized GPE (“BH-GPE”) is derivatized using N-Succinimidyl-3-(4-hydroxyphenyl)propionate (“SHPP”, Sulfo-Succinimidyl-3-(4-hydroxyphenyl)propionate (“S-SHPP”) or 3-(4-hydroxyphenyl)propionate acid hydrazide (“HPPH”).

3. The antibody of claim 1, wherein said antibody is a monoclonal antibody.

4. The antibody of claim 1, wherein said antibody is a polyclonal antibody.

5. The antibody of claim 1 having the ability to specifically bind to BH-GPE at a final titer of 1:600 using ¹²⁵I-YGPE tracer.

6. The antibody of claim 1 that is the CK5 antibody.

7. The antibody of claim 1 having the ability to specifically bind to Bolton and Hunter derivatized GPE at a final titer of 1:18,000 using Bolton and Hunter derivatized ¹²⁵I-YGPE tracer.

8. A radioimmunoassay kit for the detection or quantitation of GPE, comprising the antibody of claim 1, a GPE standard, an assay buffer, a purified GPE for iodination, a derivatizing agent for producing derivatized GPE in a sample, a second antibody or a precipitated antibody, a mixing vessel and instructions for use.

9. A radioimmunoassay kit for the detection or quantitation of GPE, comprising the antibody of claim 1, a GPE standard, an assay buffer, tritiated GPE, a derivatizing agent for producing derivatized GPE in a sample, and a second antibody or a precipitated antibody, a mixing vessel and instructions for use.

10. A radioimmunoassay kit for the detection or quantitation of GPE, comprising the antibody of claim 1, a GPE standard, Bolton and Hunter (BH) reagent, a derivatizing buffer, an assay buffer, Bolton and Hunter derivatized GPE for iodination, and a second antibody or a precipitated antibody, a mixing vessel and instructions for use.

11. The kit of claim 10, wherein said BH reagent is selected from the group consisting of SHPP, S-SHPP and HPPH.

11. A method for assaying a weakly-immunogenic molecule (“WIM”), comprising:
(a) conjugating said WIM with a conjugating agent greater than about 200 Daltons to produce a WIM conjugate;
(b) injecting said WIM conjugate into an animal capable of mounting an antibody response to said WIM conjugate;
(c) providing a sample containing said WIM;
(d) derivatizing said WIM with a derivatizing agent chemically similar to said conjugating agent but having a molecular weight of less than about 200 Daltons, thereby producing a derivatized WIM; and
(e) detecting a complex of said antibody and said derivatized WIM.

12. The method of claim 11, wherein said derivatizing agent is selected from the group consisting of BH reagent, t-boc Iodotyrosine, SHPP, S-SHPP and HPPH.

13. The method of claim 11, wherein said WIM is GPE.


SEQUENCE LISTING (7 sequences)

SEQ ID NO: 1 - protein, 3 aa, Human
GPE

SEQ ID NO: 2 - protein, 2 aa, Artificial Sequence
  fragment of human IGF-1  (1-2) IGF-1
GP

SEQ ID NO: 3 - protein, 2 aa, Artificial Sequence
  fragment of human IGF-1  (2-3) IFG-1
PE

SEQ ID NO: 4 - protein, 4 aa, Artificial Sequence
  Modified (1-3) IGF-1
YGPE

SEQ ID NO: 5 - protein, 7 aa, Artificial Sequence
  Modified (1-3) IGF-1
KYFGGPE

SEQ ID NO: 6 - protein, 3 aa, Artificial Sequence
  Modified (1-3) IGF-1
GWM

SEQ ID NO: 7 - protein, 4 aa, Artificial Sequence
  Modified (1-3) IGF-1
CGPE